(19) United States
(12) Patent Application Publication
(10) Pub. No.: US 2015/0105758 A1
(43) Pub. Date: Apr. 16, 2015

(54) METHOD FOR ENDOSCOPIC TREATMENT

(71) Applicant: OLYMPUS MEDICAL SYSTEMS CORP., Tokyo (JP)

(72) Inventor: Makoto IGARASHI, Tokyo (JP)

(73) Assignee: OLYMPUS MEDICAL SYSTEMS CORP., Tokyo (JP)

(21) Appl. No.: 14/053,691

(22) Filed: Oct. 15, 2013

(51) Int. Cl. A61B 18/22; A61B 18/14

(52) U.S. Cl. CPC A61B 18/22; A61B 18/1445; A61B 18/14; A61B 2018/00601
USPC 606/3

(57) ABSTRACT

A method for endoscopic treatment that performs treatment under an endoscope includes irradiating a subject with first narrow band light having a predetermined peak wavelength, performing mucosal incision on a living tissue after irradiation with the first narrow band light, radiating second narrow band light having a peak wavelength in spectral characteristics in a wavelength band closer to a long wavelength side than the first narrow band light after the mucosal incision, and performing treatment other than the mucosal incision on the living tissue after radiation of the second narrow band light.

### BACKGROUND OF THE INVENTION

1. Field of the Invention

The present invention relates to a method for endoscopic treatment.

2. Description of the Related Art

Conventionally, various minimally invasive inspections and operations using an endoscope are performed in the medical field. Operators can insert an endoscope into a body cavity, observe an object, images of which are picked up by an image pickup apparatus provided at a distal end portion of an endoscope insertion portion and perform treatment on a lesioned region as required using a treatment instrument inserted in a treatment instrument channel. Surgery using an endoscope does not require abdominal operation or the like, thus having an advantage of reducing physical burden on a patient.

An endoscope apparatus is configured by including an endoscope, an image processing apparatus connected to the endoscope and an observation monitor. An image pickup device provided at the distal end portion of the endoscope insertion portion picks up an image of the lesioned region and the image is displayed on the monitor. The operator can perform diagnosis or necessary treatment while watching the image displayed on the monitor.

Furthermore, some endoscope apparatuses are able to perform special light observation using special light such as infrared light not only for normal observation using white light but also for observation of blood vessels inside.

In the case of an infrared endoscope apparatus, for example, indocyanine green (ICG) having an absorption peak characteristic in near-infrared light in the vicinity of a wavelength of 805 nm is injected as medicine into the blood of the patient. The object is then irradiated with infrared light in the vicinity of a wavelength of 805 nm and in the vicinity of 930 nm from a light source apparatus by time sharing. A signal of an object image picked up by a CCD is inputted to a processor of the infrared endoscope apparatus.

Regarding such an infrared endoscope apparatus, there is a proposal on an apparatus whose processor assigns an image in the vicinity of a wavelength of 805 nm to a green color signal (G), an image in the vicinity of a wavelength of 930 nm to a blue color signal (B), and outputs the signals to a monitor (e.g., see Japanese Patent Application Laid-Open Publication No. 2000-41942). Since the image of infrared light in the vicinity of 805 nm which is more absorbed by the ICG is assigned to the green color, the operator can observe the infrared image with good contrast when the ICG is administered.

For example, in endoscopic submucosal dissection (hereinafter, referred to as “ESD”) using an endoscope to perform incision in a mucous membrane layer where a lesioned region exists and dissect the submucosa or the like, the operator needs to check the position of a relatively thick blood vessel in the mucous membrane so as not to cut the blood vessel by an electric knife or the like, and perform treatment such as incision.

Furthermore, endoscope apparatuses using narrow band light whose center wavelength is 415 nm and 540 nm are also being put to practical use. Using an endoscope apparatus using such narrow band light allows capillary vessels in a shallow layer below the living tissue to be displayed on a monitor.

### SUMMARY OF THE INVENTION

A method for endoscopic treatment according to an aspect of the present invention is a method for endoscopic treatment that performs treatment on a subject under an endoscope, the method including irradiating the subject with first narrow band light having a predetermined peak wavelength, performing mucosal incision on a living tissue of the subject after irradiation with the first narrow band light, radiating second narrow band light having a peak wavelength in spectral characteristics in a wavelength band closer to a long wavelength side than the first narrow band light after the mucosal incision, and performing treatment other than the mucosal incision on the living tissue after radiation of the second narrow band light.

A method for endoscopic treatment according to another aspect of the present invention is a method for endoscopic treatment that performs treatment on a subject under an endoscope, the method including spraying a pigment over the subject, radiating narrow band light having a predetermined peak wavelength after spraying of the pigment, and a treatment step of performing submucosal dissection or hemostasis treatment on a living tissue of the subject after radiation of the narrow band light.

### BRIEF DESCRIPTION OF THE DRAWINGS

FIG. 1 is a configuration diagram illustrating a configuration of an endoscope apparatus used for a method for endoscopic treatment according to a first embodiment of the present invention;

FIG. 2 is a diagram illustrating a configuration of a rotating filter 14 according to the first embodiment of the present invention;

FIG. 3 is a diagram illustrating an overall processing flow in first narrow band light observation according to the first embodiment of the present invention;

FIG. 4 is a diagram illustrating light absorption characteristics of venous blood according to the first embodiment of the present invention;

FIG. 5 is a flowchart illustrating a flow example of the method for endoscopic treatment in ESD according to the first embodiment of the present invention;

FIG. 6 is a diagram illustrating the shape of a lesioned region of an LST-G according to the first embodiment of the present invention;

FIG. 7 is a cross-sectional view illustrating the shape of the lesioned region AA for illustrating the type of a large intestine LST-G according to the first embodiment of the present invention;

FIG. 8 is a diagram illustrating a local injection according to the first embodiment of the present invention;

FIG. 9 is a diagram illustrating hemostasis treatment when bleeding occurs during the local injection according to the first embodiment of the present invention;

FIG. 10 is a diagram illustrating treatment of mucosal incision according to the first embodiment of the present invention;

FIG. 11 is a diagram illustrating hemostasis treatment when bleeding occurs during mucosal incision according to the first embodiment of the present invention;

FIG. 12 is a diagram illustrating submucosal dissection treatment according to the first embodiment of the present invention;

FIG. 13 is a diagram illustrating post-operation hemostasis treatment according to the first embodiment of the present invention;

FIG. 14 is a flowchart illustrating a flow example of the method for endoscopic treatment in ESD according to a second embodiment of the present invention;

FIG. 15 is a diagram illustrating a situation in which a pigment has been sprayed over the surface of the lesioned region AA according to the second embodiment of the present invention;

FIG. 16 is a diagram illustrating a relationship between wavelength and intensity of band-limited light including narrow band light having one predetermined peak wavelength and having a broad range;

FIG. 17 is a diagram illustrating a relationship between wavelength and intensity of band-limited light including narrow band light having two predetermined peak wavelengths and having a broad range; and

FIG. 18 is a diagram illustrating a relationship between wavelength and intensity of band-limited light including narrow band light having one predetermined peak wavelength and one ray of wide band light.

### DETAILED DESCRIPTION OF THE PREFERRED EMBODIMENTS

Hereinafter, embodiments of the present invention will be described with reference to the accompanying drawings.

### First Embodiment

### 1. Configuration of Endoscope Apparatus

Hereinafter, an embodiment of the present invention will be described with reference to the accompanying drawings.

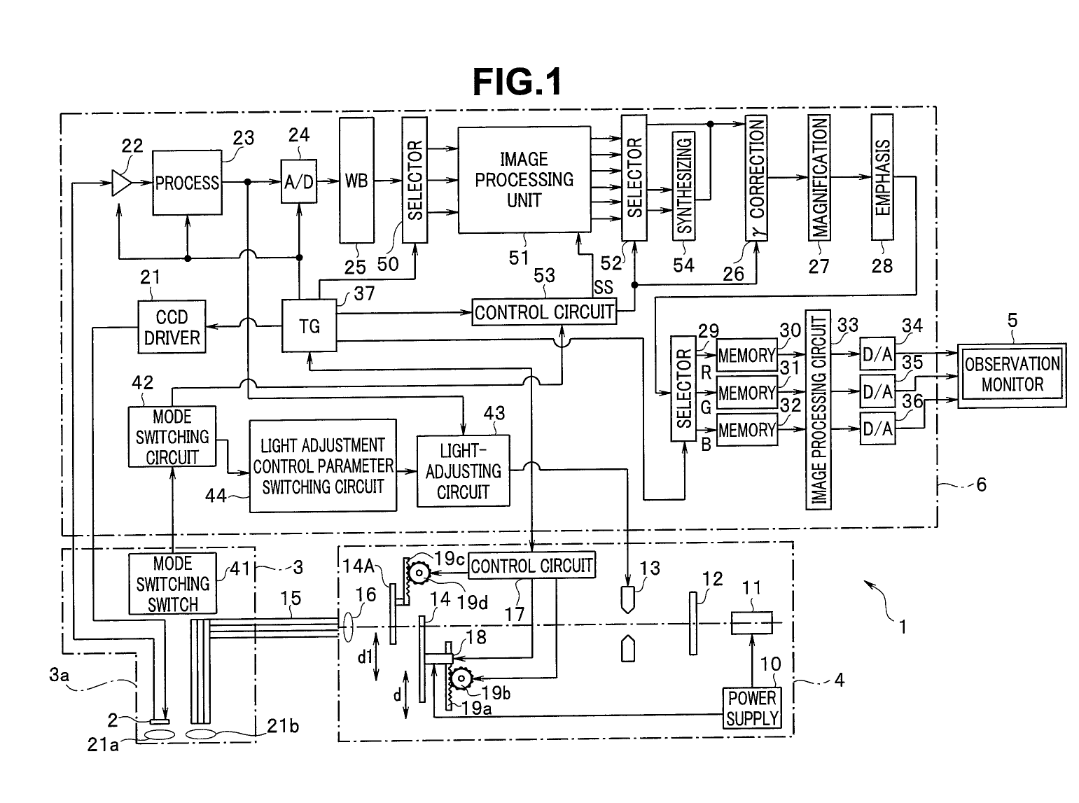

FIG. 1 is a configuration diagram illustrating a configuration of an endoscope apparatus used for a method for endoscopic treatment according to the present embodiment.

As shown in FIG. 1, an endoscope apparatus 1 of the present embodiment is constructed of an electronic endoscope 3 having a CCD 2 which is an image pickup device as a physiological image information acquiring section inserted into a body cavity to pick up an image of a tissue in the body cavity, a light source apparatus 4 that supplies illuminating light to the electronic endoscope (hereinafter, also simply referred to as “endoscope”) 3, and a video processor 6 that applies signal processing to an image pickup signal from the CCD 2 of the electronic endoscope 3 and displays an endoscopic image on an observation monitor 5. The endoscope apparatus 1 includes three modes: a normal light observation mode and two narrow band light observation modes. Note that in the following description, since the normal light observation mode of the endoscope apparatus 1 is the same as the conventional normal light observation mode, description of the configuration of the normal light observation mode is omitted, and mainly two narrow band light observation modes, that is, a first narrow band light observation mode and a second narrow band light observation mode will be described.

The endoscope 3 includes an elongated insertion portion 3a and a bending portion (not shown) is provided on a distal end side of the insertion portion 3a. The insertion portion 3a includes a distal end rigid portion on a distal end side of the bending portion and the distal end rigid portion is provided with the CCD 2. The CCD 2 constitutes an image pickup section that receives returning light of illuminating light radiated onto a subject and picks up an image of the subject. A forceps channel is provided in the insertion portion as a treatment instrument insertion channel.

The light source apparatus 4 as an illumination section is configured by including a xenon lamp 11 that emits illuminating light (white light), a heat radiation cut filter 12 that cuts heat radiation of the white light, a diaphragm apparatus 13 that controls light quantity of the white light via the heat radiation cut filter 12, a rotating filter 14 as a band-limiting section that transforms the illuminating light into frame-sequential light, a filter 14A for one narrow band light observation mode (second narrow band light observation mode) of two narrow band light observation modes, a condensing lens 16 that condenses the frame-sequential light onto a plane of incidence of a light guide 15 provided in the endoscope 3 via the rotating filter 14 and a control circuit 17 that controls the rotation and position of the rotating filter 14 and controls the position of the filter 14A. The xenon lamp 11, the rotating filter 14 and the light guide 15 constitute an irradiation section that irradiates the subject with illuminating light.

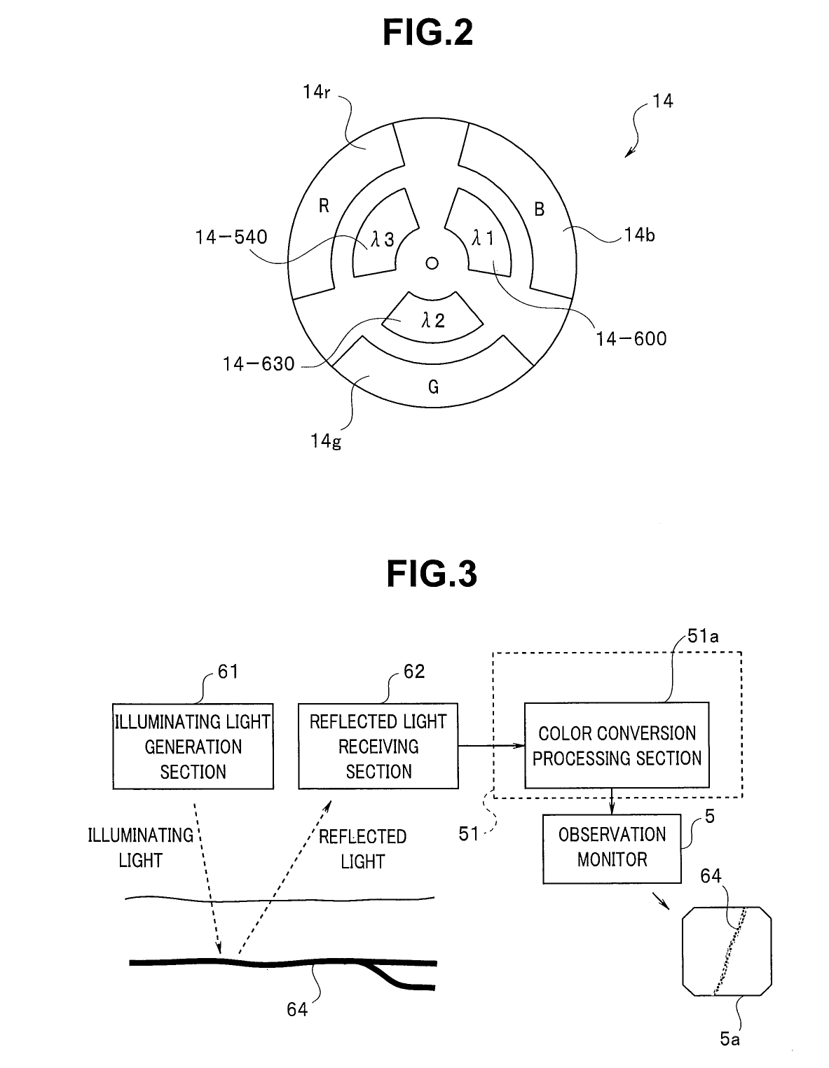

FIG. 2 is a diagram illustrating a configuration of the rotating filter 14. The rotating filter 14 is a filter that allows light from the xenon lamp 11 which is a light source to pass therethrough. The rotating filter 14 as a wavelength band-limiting section is configured into a disk shape as shown in FIG. 2, has a structure whose center is the axis of rotation and includes two filter groups. An R (red) filter section 14r, a G (green) filter section 14g and a B (blue) filter section 14b constituting a filter set to output frame-sequential light having spectral characteristics for normal light observation are arranged along a circumferential direction on an outer circumferential side of the rotating filter 14 as a first filter group.

Note that the first filter group is used when the observation mode is not only the normal light observation mode but also the first narrow band light observation mode.

Three filters 14-600, 14-630 and 14-540 that allow three light beams of predetermined narrow band wavelengths to pass therethrough are arranged along a circumferential direction on an inner circumferential side of the rotating filter 14 as a second filter group.

The filter 14-600 is configured so as to allow narrow band light in the vicinity of a wavelength of 600 nm (λ1) to pass therethrough as band-limited light. The filter 14-630 is configured so as to allow narrow band light in the vicinity of a wavelength of 630 nm (λ2) to pass therethrough as band-limited light. The filter 14-540 is configured so as to allow narrow band light in the vicinity of a wavelength of 540 nm (λ3) to pass therethrough as band-limited light.

Here, the term “vicinity” in the case of in the vicinity of a wavelength of 600 nm means narrow band light having a center wavelength of 600 nm and a width with a range of distribution of, for example, 20 nm centered on the wavelength of 600 nm (that is, from wavelength 590 nm to 610 nm around the wavelength of 600 nm). The same applies to the other wavelengths: wavelength 630 nm and wavelength 540 nm which will be described later.

The rotating filter 14 is arranged on an optical path from the xenon lamp 11 which is an illuminating light emitting section to an image pickup surface of the CCD 2 to place a limit on at least one (three here) of a plurality of wavelength bands of the illuminating light in each mode so as to narrow the wavelength bands.

The control circuit 17 then controls a motor 18 to rotate the rotating filter 14 and controls the rotation of the rotating filter 14.

A rack 19a is connected to the motor 18, a motor (not shown) is connected to a pinion 19b, and the rack 19a is threadably mounted on the pinion 19b. The control circuit 17 controls the rotation of the motor connected to the pinion 19b, and can thereby move the rotating filter 14 in a direction shown by an arrow d. Thus, the control circuit 17 controls the motor connected to the pinion 19b so as to place the first filter group in the normal light observation mode and the first filter group in the first narrow band light observation mode on an optical path in accordance with a mode switching operation by a user, which will be described later.

Furthermore, the filter 14A is a bimodal filter which allows light in the vicinity of a wavelength of 415 nm and light in the vicinity of a wavelength of 540 nm to pass therethrough. A rack 19c is connected to the filter 14A, a motor (not shown) is connected to a pinion 19d and a rack 19c is threadably mounted on the pinion 19d. The control circuit 17 controls the rotation of the motor connected to the pinion 19d, and can thereby move the filter 14A in a direction shown by an arrow d1.

Thus, the control circuit 17, according to mode switching operation by a user, which will be described later, controls the motor connected to the pinion 19d so as to place the filter 14A in an optical path in the first narrow band light observation mode and controls the motor connected to the pinion 19d so as to place the filter 14A outside the optical path in the normal light observation mode and the second narrow band light observation mode.

That is, when the observation mode is the first narrow band light observation mode, only light in the vicinity of a wavelength of 415 nm of the light that has passed through the B (blue) filter section 14b of the first filter group of the rotating filter 14 is allowed to transmit and only light in the vicinity of a wavelength of 540 nm of the light that has passed through the G (green) filter section 14g is allowed to transmit.

Note that power is supplied to the xenon lamp 11, the diaphragm apparatus 13, the rotating filter motor 18 and the motor (not shown) connected to the pinion 19b from a power supply section 10.

Thus, the light source apparatus 4 constitutes an illumination section that irradiates the subject with at least one or more illuminating light beams (two band-limited light beams, here) having predetermined wavelength bands in the first narrow band light observation mode and irradiates the subject with at least two or more illuminating light beams (three band-limited light beams, here) having predetermined wavelength bands in the second narrow band light observation mode. Here, one of the three illuminating light beams in the second narrow band light observation mode is a narrow band light beam to clearly display a blood vessel in a depth of 1 to 2 mm from a surface layer portion of a mucous membrane, and the remaining two are a narrow band light beam to display a deeper blood vessel and a narrow band light beam to display a capillary vessel in a range near the surface layer portion. For this reason, the light source apparatus 4 is an illumination apparatus that radiates at least one or more illuminating light beams via the band-limiting section that limits light to a second wavelength band (which will be described later) in the second narrow band light observation mode.

The video processor 6 is configured by including a CCD drive circuit 21 which is a CCD driver, an amplifier 22, a process circuit 23, an A/D converter 24, a white balance circuit (hereinafter, referred to as “W.B”) 25, a selector 50, an image processing unit 51, a selector 52, a γ correction circuit 26, a magnification circuit 27, an emphasis circuit 28, a selector 29, synchronization memories 30, 31 and 32, an image processing circuit 33, D/A converters 34,35 and 36, a timing generator (hereinafter, referred to as “T.G”) 37, a mode switching circuit 42, a light-adjusting circuit 43, a light adjustment control parameter switching circuit 44, a control circuit 53, and a synthesizing circuit 54 as a display image generation section.

The CCD drive circuit 21 is intended to drive the CCD 2 provided in the endoscope 3 and output a frame-sequential image pickup signal synchronized with the rotation of the rotating filter 14 to the CCD 2. Furthermore, the amplifier 22 is intended to amplify a frame-sequential image pickup signal obtained by the CCD 2 picking up an image of a tissue in the body cavity via an objective optical system 21a provided at a distal end of the endoscope 3. Furthermore, an illumination optical system 21b is provided on a distal end side of the light guide 15.

Note that polarizing plates in a crossed Nichol state may be arranged on a front surface of the CCD 2 which is an image pickup device and on a front surface of the light guide 15 respectively. The two polarizing plates in a crossed Nichol state allow the CCD 2 to pick up an image of only light from a mucous membrane depth without receiving reflected light from the mucous membrane surface.

The process circuit 23 performs correlated double sampling and noise cancellation or the like on the frame-sequential image pickup signal via the amplifier 22. The A/D converter 24 converts the frame-sequential image pickup signal that has passed through the process circuit 23 to a digital frame-sequential image signal.

The W.B 25 performs gain adjustment and white balance processing on the frame-sequential image signal digitized by the A/D converter 24 so that the brightness of an R signal of the image signal is equivalent to the brightness of a B signal of the image signal with reference to a G signal of the image signal, for example.

Note that the white balance adjustment in the W.B 25 is performed with reference to the luminance of returning light of narrow band light in the vicinity of a wavelength of 600 nm.

The selector 50 divides and outputs the frame-sequential image signal from the W.B 25 into respective sections in the image processing unit 51.

The image processing unit 51 is an image signal processing section that converts an RGB image signal for normal light observation or three or two image signals for narrow band light observation from the selector 50 to image signals for display. The image processing unit 51 outputs image signals in a normal light observation mode and respective narrow band light observation modes to the selector 52 according to a selection signal SS from the control circuit 53 based on a mode signal.

The selector 52 sequentially outputs the image signal for normal light observation and the respective image signals for narrow band light observation from the image processing unit 51 to the γ correction circuit 26 and the synthesizing circuit 54.

The γ correction circuit 26 applies γ correction processing to the frame-sequential image signal from the selector 52 or the synthesizing circuit 54. The magnification circuit 27 performs magnification processing on the frame-sequential image signal subjected to the γ correction processing in the γ correction circuit 26. The emphasis circuit 28 applies contour emphasis processing to the frame-sequential image signal subjected to the magnification processing in the magnification circuit 27. The selector 29 and the synchronization memories 30, 31 and 32 are intended to synchronize the frame-sequential image signals from the emphasis circuit 28.

The image processing circuit 33 reads the respective frame-sequential image signals stored in the synchronization memories 30, 31 and 32 and performs moving image color drift correction processing or the like. The D/A converters 34, 35 and 36 convert the image signals from the image processing circuit 33 to RGB analog video signals and outputs the signals to the observation monitor 5. The T.G 37 receives a synchronization signal which is synchronized with the rotation of the rotating filter 14 from the control circuit 17 of the light source apparatus 4 and outputs various timing signals to the respective circuits in the video processor 6.

Furthermore, the endoscope 3 is provided with a mode switching switch 41 for switching among the normal light observation mode and the two narrow band light observation modes, and the output of the mode switching switch 41 is designed to be outputted to the mode switching circuit 42 in the video processor 6. The mode switching circuit 42 of the video processor 6 is designed to output a control signal to the light adjustment control parameter switching circuit 44 and the control circuit 53. The light-adjusting circuit 43 is designed to control the diaphragm apparatus 13 of the light source apparatus 4 based on a light adjustment control parameter from the light adjustment control parameter switching circuit 44 and the image pickup signal after passing through the process circuit 23 to perform appropriate brightness control.

The respective circuits in the video processor 6 perform predetermined processing in accordance with a specified mode. Those circuits perform processing in accordance with the normal light observation mode and the two narrow band light observation modes respectively, and the observation monitor 5 displays an image for normal light observation or an image for narrow band light observation. As will be described later, in the first narrow band light observation mode, the observation monitor 5 displays an image based on an image signal of a relatively thick blood vessel having a diameter on the order of 1 to 2 mm at a depth of the mucous membrane on the order of 1 to 2 mm from the surface layer portion of the mucous membrane.

### 2. Overall Processing Flow of First and Second Narrow Band Light Observations

First, an overall approximate flow of second narrow band light observation according to the present embodiment will be described briefly.

FIG. 3 is a diagram illustrating an overall processing flow in second narrow band light observation according to the present embodiment.

The operator inserts the insertion portion of the endoscope into the body cavity and places the distal end portion of the endoscope insertion portion in the vicinity of a lesioned region in a normal light observation mode. To observe a relatively thick blood vessel of, for example, 1 to 2 mm in diameter, in the depth running through the muscularis propria from the submucosa, the operator operates the mode switching switch 41 to switch the observation mode of the endoscope apparatus 1 to the second narrow band light observation mode.

In the second narrow band light observation mode, the control circuit 17 of the endoscope apparatus 1 controls the motor connected to the pinion 19b to move the position of the rotating filter 14 so as to emit light that has passed through the second filter group from the light source apparatus 4. The control circuit 53 also controls the various circuits in the video processor 6 so as to perform image processing for observation using a narrow band wavelength.

As shown in FIG. 3, in the second narrow band light observation mode, illuminating light having a narrow band wavelength is emitted from an illuminating light generation section 61 from the distal end portion of the insertion portion of the endoscope 3, and after passing through the mucous membrane layer, radiated onto the blood vessel 64 running through the submucosa and the muscularis propria. Here, the illuminating light generation section 61 is configured by including the light source apparatus 4, the rotating filter 14 and the light guide 15 or the like, and emits illuminating light from the distal end of the endoscope insertion portion. As the rotating filter 14 rotates, narrow band light in the vicinity of a wavelength of 600 nm, narrow band light in the vicinity of a wavelength of 630 nm and narrow band light in the vicinity of a wavelength of 540 nm are consecutively and sequentially emitted from the light source apparatus 4 as band-limited light beams and radiated onto the object.

Reflected light beams of the narrow band light in the vicinity of a wavelength of 600 nm, the narrow band light in the vicinity of a wavelength of 630 nm and the narrow band light in the vicinity of a wavelength of 540 nm are respectively received by a reflected light receiving section 62 which is the CCD 2. The CCD 2 outputs image pickup signals of the respective reflected light beams and supplies the image pickup signals to the selector 50 via the amplifier 22 or the like. The selector 50 maintains a first image signal P1 in the vicinity of a wavelength of 600 nm, a second image signal P2 in the vicinity of a wavelength of 630 nm and a third image signal P3 in the vicinity of a wavelength of 540 nm in accordance with predetermined timing from the T.G 37 and supplies the image signals to the image processing unit 51. The image processing unit 51 includes a color conversion processing section 51a for the narrow band light observation mode.

The operator can set the endoscope apparatus 1 to the second narrow band light observation mode to cause the relatively thick blood vessel in the depth of the mucous membrane to be displayed on a screen 5a of the observation monitor 5 as shown in FIG. 3 in, for example, red color or magenta color with relatively high contrast.

Furthermore, the operator can also set the endoscope apparatus 1 to the second narrow band light observation mode to cause not only the blood vessel below the surface of a living tissue but also a bleeding point at which bleeding has occurred to be drawn on the observation monitor 5. This is because even when bleeding occurs from the bleeding point on the mucous membrane surface of the mucous membrane and the mucous membrane surface is covered with the blood, when the blood is observed in the second narrow band light observation mode, narrow band light in the vicinity of a wavelength of 600 nm passes through the blood and the blood running from the bleeding point on the mucous membrane surface is displayed on the observation monitor 5. Since a variation in a density (that is, concentration) of the blood flowing from the bleeding point or a variation in the thickness of the blood layer is high in the vicinity of the bleeding point, the flow of the blood flowing from the bleeding point is visualized so that the operator can visually recognize the blood flow, identify the bleeding point below the blood and the operator can speedily apply hemostasis treatment to the bleeding point.

Therefore, the color conversion processing section 51a of the image processing unit 51 in FIG. 1 assigns the respective image signals to respective channels of RGB of the observation monitor 5 and supplies the image signals to the selector 52. As a result, the relatively thick blood vessel 64 in the depth of the mucous membrane and the bleeding point at which bleeding has occurred are displayed on the screen 5a of the observation monitor 5 with high contrast as shown in FIG. 3.

For example, in order for the color conversion processing section 51a to display a blood vessel 64 in the depth with high contrast using narrow band light NL1 in the vicinity of a wavelength of 600 nm, the color conversion processing section 51a assigns the first image signal P1 (λ1), the second image signal P2 (λ2) and the third image signal P3 (λ3) to the G, R and B channels respectively.

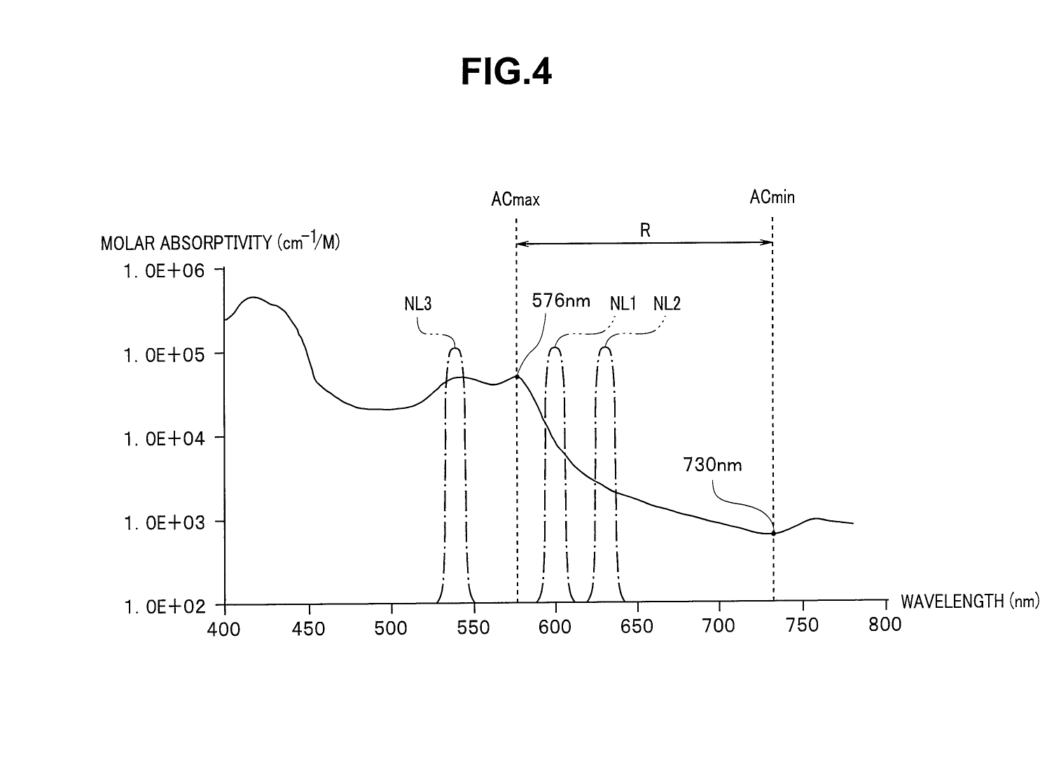

Here, light absorption characteristics of venous blood will be described. FIG. 4 is a diagram illustrating light absorption characteristics of venous blood. The vertical axis in FIG. 4 shows molar absorptivity (cm⁻¹/M) and the horizontal axis shows wavelength. Note that although three narrow band illuminating light beams are affected by scattering characteristics of the living tissue itself, the scattering characteristics of the living tissue itself monotonously decrease as the wavelength increases, and therefore FIG. 4 will be described as the absorption characteristics of the living tissue.

Generally, the venous blood contains oxygenated hemoglobin (HbO₂) and reduced hemoglobin (Hb) (hereinafter, both will be simply jointly referred to as “hemoglobin”) at a proportion of 60:40. Light is absorbed by hemoglobin, but the absorption coefficient thereof varies from one wavelength of light to another. FIG. 4 shows light absorption characteristics of venous blood for each wavelength from 400 nm to approximately 800 nm, and the absorptivity shows a maximum value at a point of wavelength of approximately 576 nm and a minimum value at a point of wavelength of 730 nm in a range from 550 nm to 750 nm.

In the second narrow band light observation mode, three narrow band light beams are radiated and their respective returning light beams are received by the CCD 2.

The narrow band light in the vicinity of a wavelength of 600 nm (hereinafter referred to as “first narrow band light NL1”) is light in a wavelength band within a wavelength band R from a maximum value ACmax (here, absorptivity at a wavelength of 576 nm) to a minimum value ACmin (here, absorptivity at a wavelength of 730 nm) of absorption characteristics of hemoglobin.

The narrow band light in the vicinity of a wavelength of 630 nm (hereinafter, also referred to as “second narrow band light NL2”) is also light within the wavelength band R from the maximum value ACmax to the minimum value ACmin of absorption characteristics of hemoglobin, but it is light in a wavelength band having a longer wavelength than the first narrow band light NL1, lower absorptivity and with suppressed scattering characteristics of a living tissue. The suppressed scattering characteristics mean that the scattering coefficient decreases toward the long wavelength side.

That is, the light source apparatus 4 radiates first illuminating light NL1 having a peak wavelength in spectral characteristics between the wavelength band including the maximum value ACmax and the wavelength band including the minimum value ACmin in the absorption characteristics of the living tissue.

Furthermore, the light source apparatus 4 also radiates second illuminating light NL2 having lower absorption characteristic values than the image signal P1 resulting from the first illuminating light NL1 and having a peak wavelength in spectral characteristics with suppressed scattering characteristics of the living tissue.

Moreover, the light source apparatus 4 also radiates narrow band light in the vicinity of a wavelength of 540 nm (hereinafter, referred to as “third narrow band light NL3”). The third narrow band light NL3 is light in a wavelength band other than the wavelength band R from the maximum value ACmax to the minimum value ACmin in the absorption characteristics of hemoglobin and is illuminating light transmittable by a predetermined distance from the surface layer portion of the mucous membrane surface of the subject.

The CCD 2 outputs image pickup signals of the respective images of three narrow band light beams. Thus, each image includes a plurality of pixel signals based on respective returning light beams of the first, second and third narrow band light beams NL1, NL2 and NL3.

The first narrow band light NL1 and the second narrow band light NL2 repeat multiple scattering processes in the living tissue respectively, and are consequently emitted from the mucous membrane surface as returning light. The first narrow band light NL1 and the second narrow band light NL2 have their respective mean free paths. The mean free path of the first narrow band light NL1 is shorter than the mean free path of the second narrow band light NL2.

Thus, the first narrow band light NL1 in the vicinity of a wavelength of 600 nm (λ1) reaches the vicinity of the blood vessel 64 and the second narrow band light NL2 in the vicinity of a wavelength of 630 nm (λ2) reaches a position slightly deeper than the blood vessel 64. Using this first narrow band light NL1 thereby makes it possible to display a relatively thick blood vessel having a diameter of 1 to 2 mm and a bleeding point at which bleeding has occurred, located in a relatively deep part, 1 to 2 mm below the surface layer of the mucous membrane of the living body.

The second narrow band light NL2 in the vicinity of a wavelength of 630 nm (λ2) also makes it possible to display a thicker blood vessel and a bleeding point at which bleeding has occurred, located in a deeper part.

Here, although the narrow band light NL1 or NL2 is light in the aforementioned wavelength band, the range of light in which the relatively thick blood vessel can be displayed with high contrast is from 585 nm which is the minimum wavelength to 630 nm which is the maximum wavelength.

The endoscope apparatus 1 radiates the above-described narrow band light, and can thereby cause the observation monitor 5 to display the blood vessel in the living tissue and the bleeding point at which bleeding has occurred.

Furthermore, the first narrow band light observation mode is a publicly known narrow band light observation mode, and the first narrow band light observation mode makes it possible to highlight a fine pattern of a blood vessel or mucous membrane of the mucous membrane surface using narrow band light whose center wavelength is 415 nm and narrow band light whose center wavelength is 540 nm.

As described above, using the aforementioned endoscope apparatus 1, the operator can switch from radiation of white light to radiation of narrow band light in such a way as to irradiate the subject with white light in the normal light observation mode, irradiate the subject with narrow band light having a predetermined peak wavelength in the second narrow band light observation mode or irradiate the subject with predetermined narrow band light in the first narrow band light observation mode. The narrow band light in the second narrow band light observation mode is light in a red band of a visible range including narrow band light having a peak wavelength in spectral characteristics between a wavelength band including a maximum value and a wavelength band including a minimum value in hemoglobin light absorption characteristics of the living tissue of the subject.

Hereinafter, illuminating light including three narrow band light beams (NL1, NL2, NL3) radiated in the second narrow band light observation mode is called “second narrow band illuminating light” and illuminating light including two narrow band light beams (narrow band light whose center wavelength is 415 nm and narrow band light whose center wavelength is 540 nm) radiated in the first narrow band light observation mode is called “first narrow band illuminating light.”

### 3. Flow of Endoscopic Treatment of ESD

Next, an example of the method for endoscopic treatment of the present embodiment that applies treatment to a subject under an endoscope will be described.

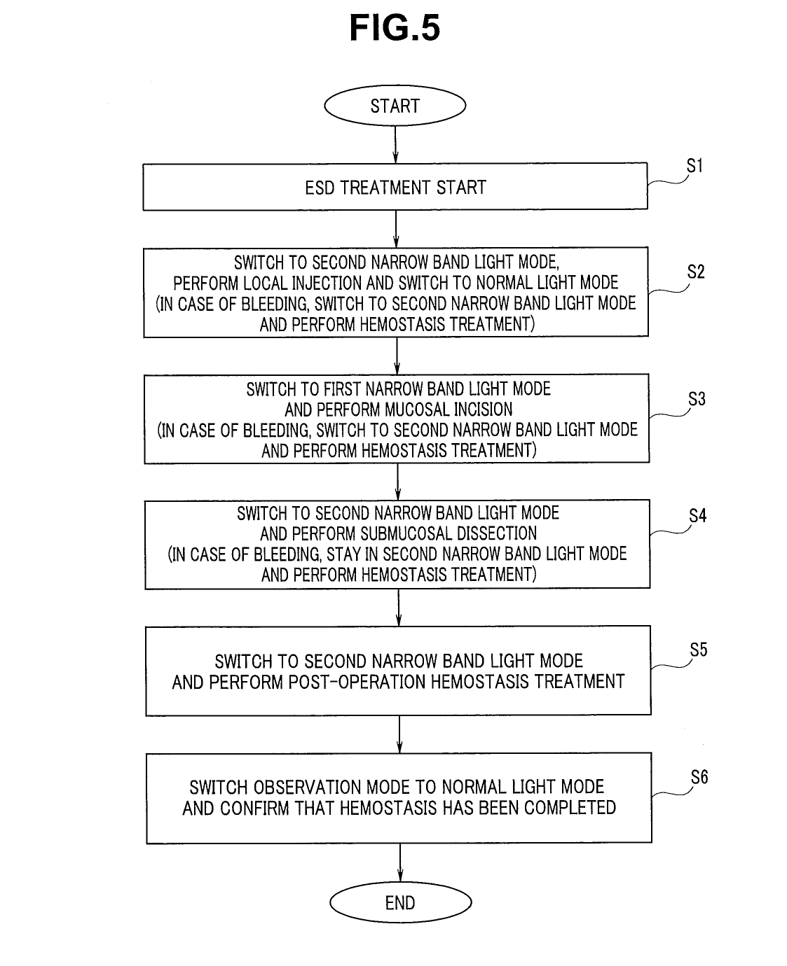

FIG. 5 is a flowchart illustrating a flow example of the method for endoscopic treatment in ESD. ESD is performed on the large intestine here. The method for endoscopic treatment of the present embodiment will be described below on a step-by-step basis.

### [ESD Treatment Start]

The operator inserts the endoscope 3 into the body of the subject by setting the observation mode which is one of operating modes of the endoscope apparatus 1 to a normal light observation mode and causes the distal end portion of the insertion portion 3a to approach the vicinity of the lesioned region by operating the bending portion while watching the image on the observation monitor 5 under white light observation.

The operator irradiates the subject with white light, visually recognizes a lesioned region and starts ESD treatment (S1).

When the lesioned region is a large intestine tumor, the surface of the lesioned region of the large intestine mucous membrane has a concavo-convex shape which is bulging relative to its periphery, and the boundary between the lesioned region and the normal mucous membrane is easily recognizable to the operator under normal white light observation. For example, since a large intestine LST-G (laterally spreading tumor granular type) has a granular surface layer, the boundary between the lesioned region and the normal mucous membrane is easily recognizable to the operator under normal white light observation.

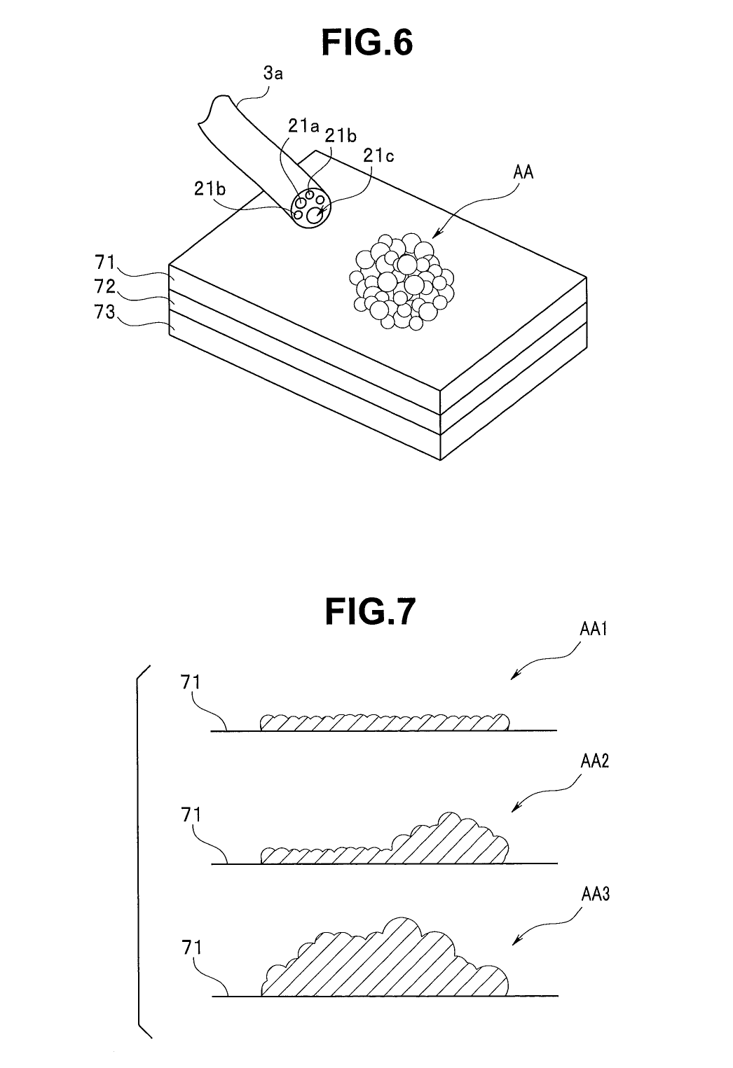

FIG. 6 is a diagram illustrating the shape of a lesioned region of an LST-G. FIG. 6 is a diagram illustrating a situation in which the distal end portion of the insertion portion 3a of the endoscope 3 is brought close to a lesioned region AA and the lesioned region AA is included in the range of field of view of the endoscope 3. Note that in FIG. 6 and subsequent diagrams, a living tissue made up of a mucous membrane layer 71, a submucosa 72 and a muscular layer 73 is represented by a partially cut out rectangular parallelepiped.

The lesioned region AA is located in the mucous membrane layer 71, the submucosa 72 is located below the mucous membrane layer 71 and the muscular layer 73 is located below the submucosa 72. The observation mode of the endoscope apparatus 1 is a normal light observation mode and the operator places the distal end portion of the endoscope 3 in the vicinity of the lesioned region AA under white light observation.

FIG. 7 is a cross-sectional view for illustrating the shape of the lesioned region AA provided for describing the type of the large intestine LST-G. FIG. 7 shows three types AA1, AA2 and AA3 as the lesioned region AA of the large intestine LST-G. The lesioned region of the type AA1 large intestine LST-G is a homogenous type, and the lesioned region of the type AA2 or AA3 large intestine LST-G is a nodular mixed type. Since both large intestine LST-G types are granular types, the boundary between the lesioned region AA and the normal mucous membrane is clearly recognizable to the operator.

### [Local Injection]

Returning to FIG. 5, the operator switches the observation mode from the normal light observation mode to the second narrow band light observation mode, performs local injection, and then switches the observation mode to the normal light observation mode (S3). The local injection is performed under second narrow band light observation.

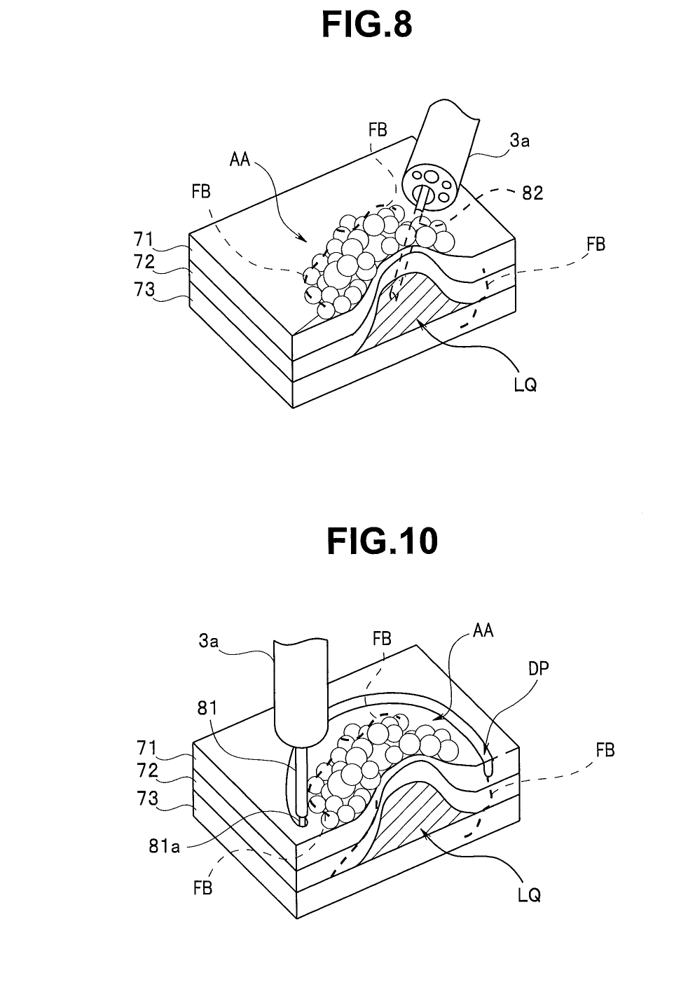

FIG. 8 is a diagram illustrating the local injection.

When the observation mode is switched to the second narrow band light observation mode, the relatively thick blood vessel in the depth of the mucous membrane is displayed in, for example, red color or magenta color. Thus, the operator passes a local injection needle 82 through the forceps channel, and can thereby inject a local injection liquid LQ (shown by the oblique lines) into the submucosa 72 while avoiding the deep blood vessel FB (shown by a dotted line) which is visually recognizable in the second narrow band light observation mode as shown in FIG. 8.

Note that when bleeding occurs during the local injection, the operator must perform hemostasis and the operation time is extended by the hemostasis time. Thus, in the second narrow band light observation mode, the operator can perform local injection while avoiding the deep blood vessel, and can thereby prevent bleeding during the operation and shorten the operation time.

Therefore, after removing the local injection needle 82 from the mucous membrane, the operator switches the observation mode from the second narrow band light observation mode to the normal light observation mode, and checks the presence or absence of bleeding. If there is no bleeding, the operator switches the observation mode from the normal light observation mode to the first narrow band light observation mode and shifts to the next mucosal incision treatment.

If the blood vessel is punctured by the distal end of the local injection needle 82 and bleeding occurs during the local injection, the operator performs hemostasis treatment and checks the hemostasis results.

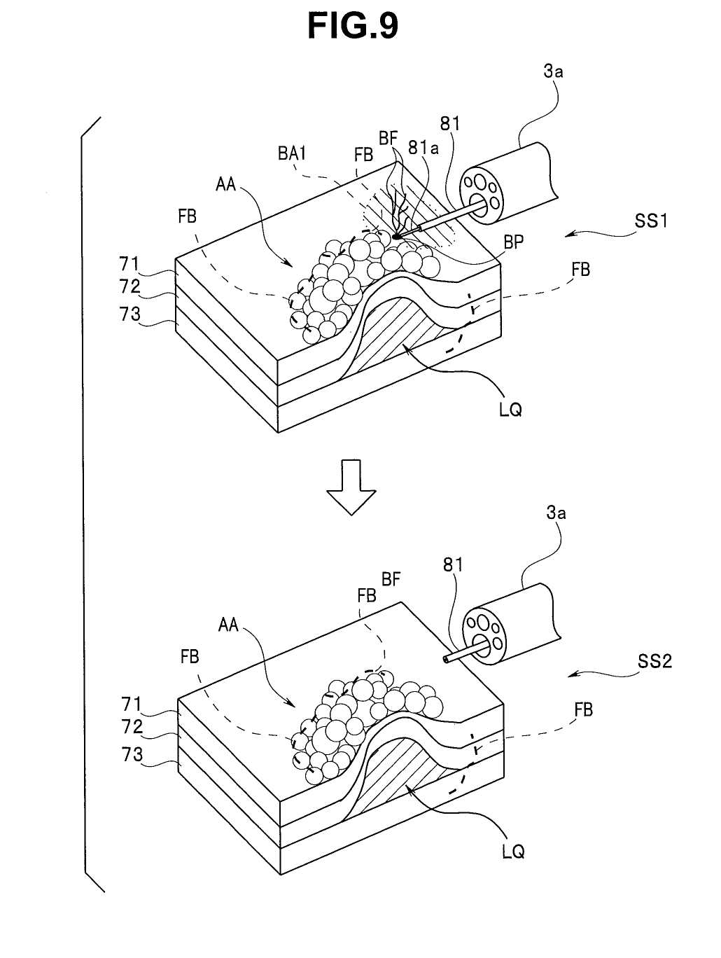

FIG. 9 is a diagram illustrating hemostasis treatment when bleeding occurs during the local injection. After the bleeding is detected, the operator can visually recognize the bleeding point from the observation monitor 5 in the second narrow band light observation mode, and can thereby recognize a bleeding flow BF in a bleeding region BA1 shown by the oblique lines and identify a bleeding point BP from the flow BF as shown in SS1.

As a result, the operator can perform hemostasis treatment using the high-frequency scalpel 81 or hemostasis forceps. In the case of the high-frequency scalpel 81, hemostasis treatment is performed by making the distal end portion 81a of the high-frequency scalpel 81 contact the bleeding point BP and passing a high-frequency current therethrough. In the case of the hemostasis forceps, hemostasis treatment is performed by grasping the bleeding point BP with the surface of the grasping portion of the distal end of the hemostasis forceps and passing a high-frequency current therethrough.

After the hemostasis treatment, the operator switches the observation mode from the second narrow band light observation mode to the normal light observation mode to thereby switch from irradiation with narrow band light to irradiation with white light and checks whether or not the hemostasis treatment has been completed as shown in SS2.

As described above, in local injection, by switching from irradiation with the white light to irradiation of the subject with the second narrow band illuminating light having a predetermined peak wavelength according to the condition of bleeding that occurs from the living tissue and radiating the second narrow band illuminating light, the operator can perform hemostasis treatment while visually recognizing the bleeding point.

As described above, the operator then switches the observation mode from the normal light observation mode to the first narrow band light observation mode and shifts to the next mucosal incision treatment.

### [Mucosal Incision]

After local injection, the operator switches the observation mode to the first narrow band light observation mode and performs mucosal incision (S3). That is, in S3, the operator irradiates the subject with narrow band light having a predetermined peak wavelength in the first narrow band light observation mode and performs mucosal incision on the living tissue of the subject after the irradiation with the narrow band light.

FIG. 10 is a diagram illustrating mucosal incision treatment. With the high-frequency scalpel 81 inserted through the forceps channel, the operator performs mucosal incision as shown in FIG. 10. That is, after radiation of the white light, the operator switches the observation mode to the first narrow band light observation mode and performs mucosal incision on the living tissue of the subject.

In the case of ESD of the stomach or the like, marking is normally applied to define the range in which mucosal incision is performed. However, in the case of ESD of the large intestine, since the large intestine is thin, marking is often not applied. Therefore, to make it easier to determine the range of mucosal incision, the operator radiates narrow band light in the first narrow band light observation mode to highlight the lesion range and performs incision on the periphery of the lesion range in mucosal incision under first narrow band illuminating light.

The mucosal incision is performed by causing the distal end portion 81a of the high-frequency scalpel 81 to contact the living tissue outside the lesioned region AA. By moving the distal end portion 81a along the outer circumferential portion within the range of the lesioned region AA, the operator can form a notch as shown in FIG. 10. An incision portion DP which is the notch formed by the distal end portion 81a of the high-frequency scalpel 81 is formed outside the lesioned region AA. FIG. 10 illustrates a situation in which the incision portion DP has been formed up to a midpoint of the outer circumferential portion of the range of the lesioned region AA.

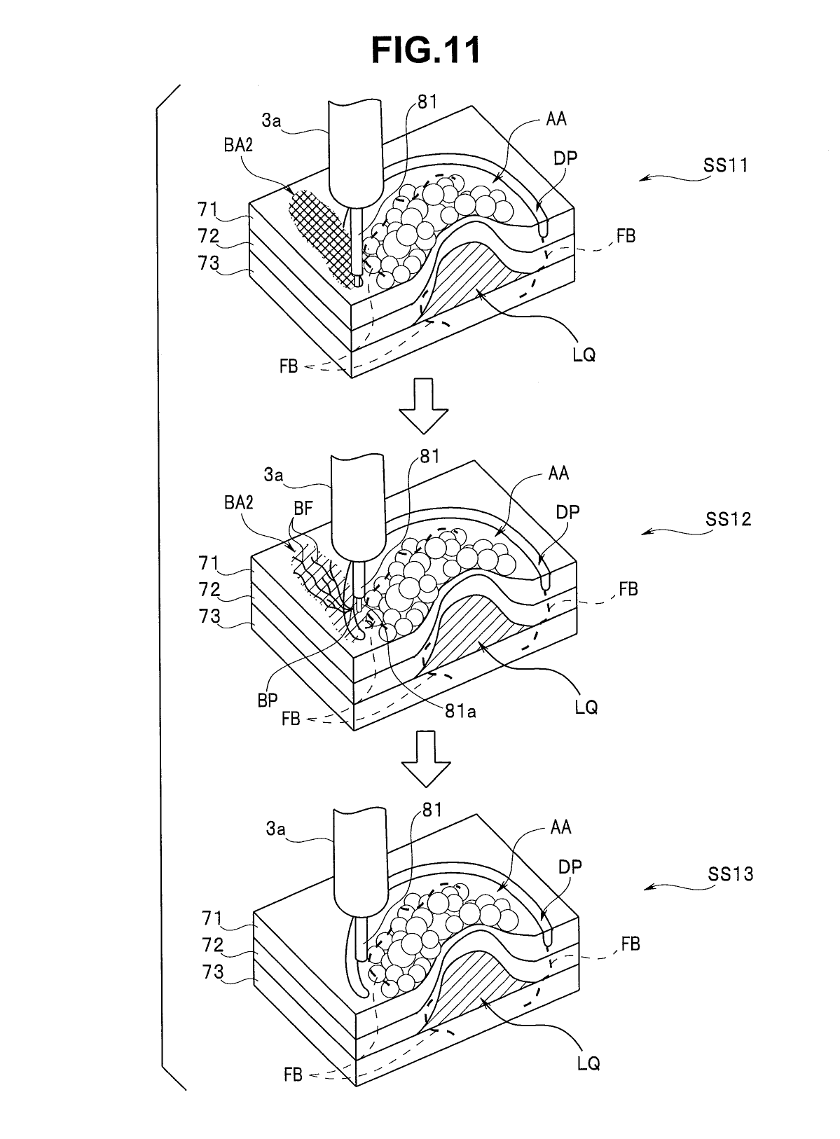

If bleeding occurs during the mucosal incision, the operator switches the observation mode from the first narrow band light observation mode to the second narrow band light observation mode and performs hemostasis treatment. FIG. 11 is a diagram illustrating hemostasis treatment when bleeding occurs during mucosal incision.

For example, as shown in SS11 in FIG. 11, when the operator damages the deep blood vessel FB (shown by a dotted line) by the distal end portion 81a of the high-frequency scalpel 81 during the mucosal incision and bleeding occurs, the operator cannot check the bleeding point BP below a bleeding region BA2 shown by a shaded area under normal observation or first narrow band light observation.

Thus, the operator switches the observation mode to the second narrow band light observation mode so as to visually recognize the bleeding point BP from a bleeding flow BF that exists below the bleeding region BA2 shown by the shaded area as shown in SS12. Upon detecting the bleeding point BP, the operator performs hemostasis using the high-frequency scalpel 81 (or hemostasis forceps). In the second narrow band light observation mode, the bleeding region BA2 is displayed, for example, in yellow color or orange color, the bleeding flow BF is displayed in dark orange color, the bleeding point BP is displayed in yellow color and the deep blood vessel FB is displayed, for example, in red color or magenta color.

After the hemostasis treatment, the operator switches the observation mode from the second narrow band light observation mode to the normal light observation mode to thereby switch from radiation of second narrow band light to radiation of white light and confirms that the hemostasis treatment has been completed as shown in SS13.

That is, in mucosal incision, the operator switches from radiation of the first narrow band light to radiation of the second narrow band illuminating light having a predetermined peak wavelength in accordance with the condition of bleeding that occurs from the living tissue, radiates the second narrow band illuminating light, and can thereby check the bleeding point and perform hemostasis treatment which is treatment other than the mucosal incision.

After the hemostasis, the operator resumes mucosal incision in the first narrow band light observation mode, performs mucosal incision around the whole circumference of the lesioned region AA and then switches the observation mode from the first narrow band light observation mode to the second narrow band light observation mode and shifts to submucosal dissection treatment.

If no bleeding occurs during mucosal incision, the observation mode is kept to the first narrow band light observation mode and not shifted to the second narrow band light observation mode.

### [Submucosal Dissection]

Returning to FIG. 5, next, the operator performs treatment of submucosal dissection (S4). Submucosal dissection is performed using the high-frequency scalpel 81 in the second narrow band light observation mode. That is, after the mucosal incision, the operator radiates narrow band light in the second narrow band light observation mode having a peak wavelength in wavelength band spectral characteristics closer to a long wavelength side than the narrow band light radiated in the first narrow band light observation mode, and after also radiating narrow band light in the second narrow band light observation mode, performs submucosal dissection treatment on the living tissue as treatment other than the mucosal incision.

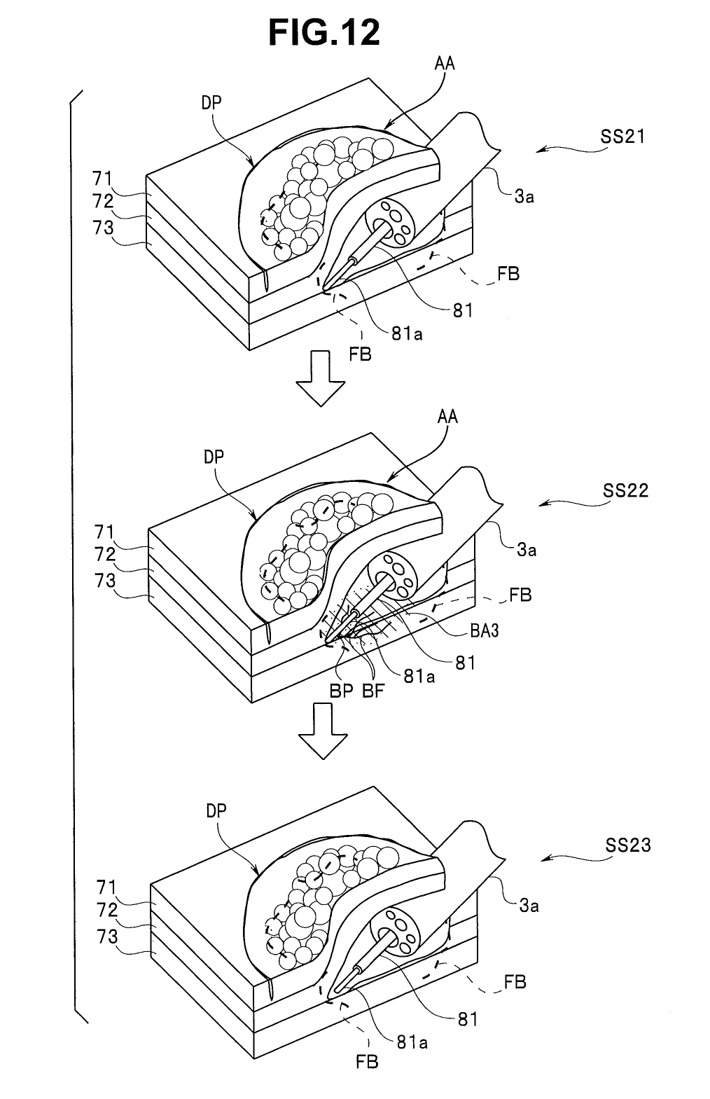

FIG. 12 is a diagram illustrating submucosal dissection treatment. As shown in SS21, submucosal dissection is performed by causing the distal end portion 81a of the high-frequency scalpel 81 to contact the submucosa 72 directly on the muscular layer 73.

If bleeding occurs during the submucosal dissection, the observation mode is kept to the second narrow band light observation mode and hemostasis treatment is performed.

As shown in SS22 in FIG. 12, if the operator damages the deep blood vessel FB by the distal end portion 81a of the high-frequency scalpel 81 during the submucosal dissection and bleeding occurs, it is difficult for the operator to check the bleeding point below a bleeding region BA3 under normal observation.

Thus, the operator keeps the observation mode to the second narrow band light observation mode and checks a bleeding point BP located below a bleeding region BA3 shown by the oblique lines as shown in SS22. When the operator can detect the bleeding point BP from a bleeding flow BF, the operator performs hemostasis using the high-frequency scalpel 81 (or hemostasis forceps).

After the hemostasis treatment, the operator switches the observation mode to the normal light observation mode to thereby switch from radiation of the second narrow band light to radiation of the white light, confirms that the hemostasis treatment has been completed as shown in SS23, switches the observation mode to the second narrow band light observation mode and resumes submucosal dissection.

That is, in submucosal dissection treatment, the operator performs hemostasis treatment after checking the bleeding point in the second narrow band light observation mode having a predetermined peak wavelength according to the condition of bleeding that occurs from the living tissue.

### [Post-Operation Hemostasis]

The operator then performs post-operation hemostasis treatment (S5). Upon completion of the submucosal dissection treatment, the operator keeps the observation mode to the second narrow band light observation mode, checks the deep blood vessel FB located in the vicinity of an incision surface DS and coagulates the deep blood vessel FB located in the vicinity of the surface of the incision surface DS using the high-frequency scalpel 81. That is, after the submucosal dissection treatment, the operator performs preventive hemostasis treatment which is treatment other than the mucosal incision on the living tissue while radiating the second narrow band illuminating light.

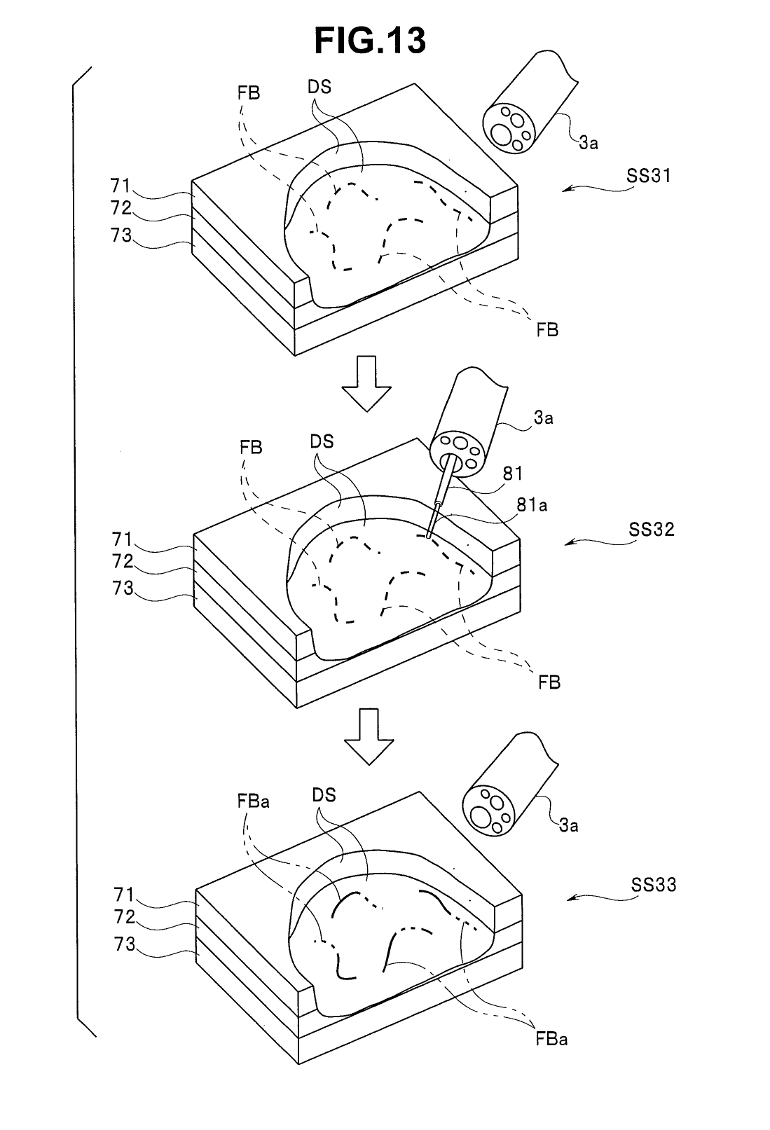

FIG. 13 is a diagram illustrating the post-operation hemostasis treatment. In SS31 and SS32 in FIG. 13, the deep blood vessel FB located in the vicinity of the incision surface DS is shown by a dotted line. The deep blood vessel FB is located in the vicinity of the incision surface DS of the portion from which the mucous membrane layer 71 and the submucosa 72 have been removed by submucosal dissection.

Since the incision surface DS by submucosal dissection treatment appears in red color under white color normal light observation, it is difficult for the operator to visually recognize the deep blood vessel FB beneath the incision surface DS. After the operation, since bleeding is likely to occur from the deep blood vessel FB beneath the incision surface DS, it is desirable to coagulate the deep blood vessel FB located in the vicinity of the surface of the incision surface DS.

After the submucosal dissection treatment, the operator switches the observation mode to the second narrow band light observation mode and causes the observation monitor 5 to display the deep blood vessel FB located in the vicinity of the incision surface DS in red color or magenta color to check the position of the deep blood vessel FB.

Next, the operator causes the distal end portion 81a of the high-frequency scalpel 81 to contact the incision surface DS on the detected deep blood vessel FB or causes the detected thick blood vessel FB to be grasped with the surface of the grasping portion at the distal end of the hemostasis forceps.

As shown in SS32, by passing a high-frequency current through the distal end portion 81a of the high-frequency scalpel 81 or the distal end of the hemostasis forceps, the operator can cause the deep blood vessel FB in the vicinity of the incision surface DS to coagulate and perform preventive hemostasis. Post-operation hemostasis treatment is applied to the whole deep blood vessel FB located in the vicinity of the incision surface DS. As shown in SS33 in FIG. 13, the deep blood vessel FB subjected to the coagulation treatment is shown by a two-dot dashed line.

Upon completion of the coagulation treatment on the deep blood vessel FB, the operator switches the observation mode from the second narrow band light observation mode to the normal light observation mode, observes the whole treatment region as shown in SS33, makes sure that there is no bleeding and removes, when there is no bleeding, the insertion portion 3a of the endoscope 3 from the inside of the body.

As described above, according to the aforementioned embodiment, the operator can perform appropriate mucosal incision in ESD in the large intestine and can also speedily perform hemostasis treatment when bleeding occurs in mucosal incision and treatment other than the mucosal incision.

### Second Embodiment

Next, a second embodiment will be described. In the first embodiment, the subject is irradiated with first narrow band illuminating light having a predetermined peak wavelength, mucosal incision is then performed on the living tissue, second narrow band illuminating light having a peak wavelength in spectral characteristics in a wavelength band closer to a long wavelength side than the first narrow band light is radiated and treatment other than mucosal incision is performed on the living tissue after radiation of the second narrow band illuminating light. In the second embodiment, however, mucosal incision is performed after spraying a pigment.

Since an endoscope apparatus used for a method for endoscopic treatment according to the second embodiment is similar to the endoscope apparatus 1 described in the first embodiment, description of the configuration of the apparatus is omitted and the method for endoscopic treatment of the second embodiment will be described. Note that the method for endoscopic treatment of the first embodiment uses three observation modes: normal light observation mode, first narrow band light observation mode and second narrow band light observation mode, whereas the method for endoscopic treatment of the second embodiment uses two observation modes: normal light observation mode and second narrow band light observation mode, and therefore the endoscope apparatus of the second embodiment may be an endoscope apparatus resulting from omitting the function of the first narrow band light observation mode from the endoscope apparatus 1 described in the first embodiment.

In addition, the method for endoscopic treatment also includes the same steps as those of the first embodiment, and therefore the same treatment steps will be assigned the same reference numerals and description thereof will be simplified and different steps will be described in detail.

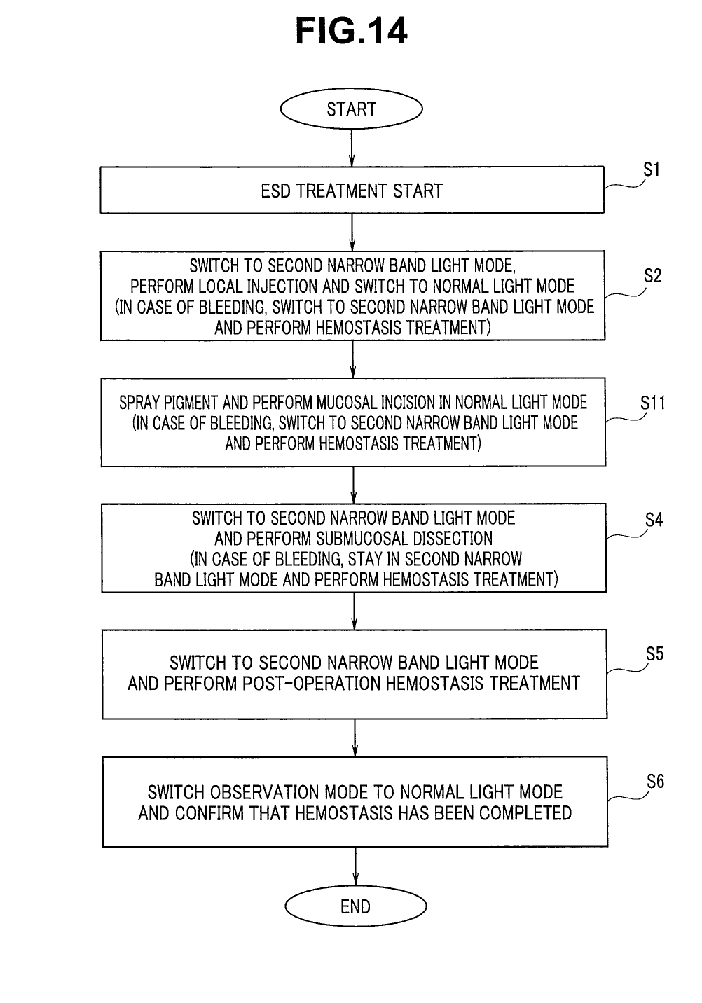

FIG. 14 is a flowchart illustrating a flow example of the method for endoscopic treatment in ESD. ESD is also performed on the large intestine or the like as in the case of the first embodiment.

In FIG. 14, S1 and S2 are the same treatments as those in S1 and S2 in FIG. 5, and after starting ESD treatment (S1), the operator switches to the second narrow band light mode, performs local injection and switches the observation mode to the normal light observation mode after the local injection. If bleeding occurs due to the local injection, the operator switches to the second narrow band light observation mode and performs hemostasis treatment (S2).

After the local injection, the operator sprays a pigment over the lesioned region AA, and after the spray of pigment, performs mucosal incision in the normal light observation mode (S11).

As described above, in the case of ESD of the large intestine, the large intestine is thin, and so marking is not performed. Thus, in the present embodiment, a pigment Pg is sprayed over the subject to make it easier to determine the range of mucosal incision and mucosal incision is performed under normal light observation.

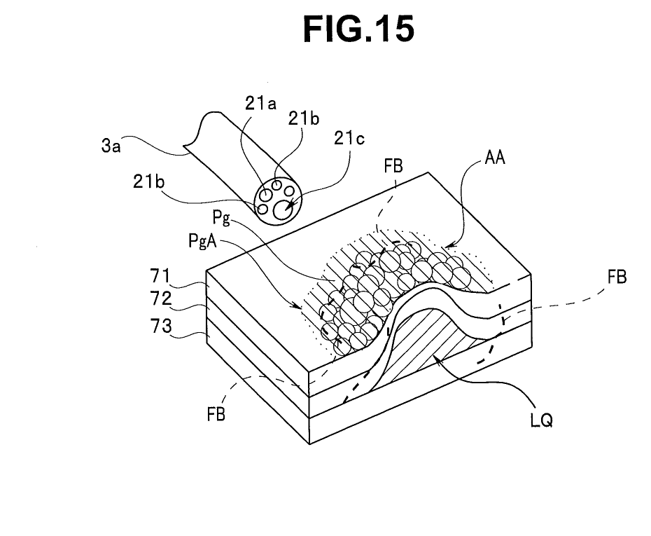

FIG. 15 is a diagram illustrating a situation in which the pigment has been sprayed over the surface of the lesioned region AA.

The operator sprays the pigment Pg over the lesioned region AA from an opening 21c of the treatment instrument at the distal end portion of the insertion portion 3a of the endoscope 3 via the forceps channel provided in the insertion portion of the endoscope 3. In FIG. 15, a spray range PgA where the pigment Pg has been sprayed is the range shown by oblique lines. The pigment Pg is indigo carmine or indigo carmine and acetic acid. The spray of the pigment Pg allows the operator to clearly observe the boundary between the normal mucous membrane and the lesioned region AA displayed on the observation monitor 5 and thereby to perform mucosal incision treatment more easily.

As described above, in the present embodiment, the operator sprays the pigment Pg and performs mucosal incision treatment in the normal light observation mode.

If bleeding occurs during the mucosal incision treatment, the operator switches the observation mode from the normal light observation mode to the second narrow band light observation mode and performs hemostasis treatment after radiation of narrow band light in the second narrow band light observation mode. Hemostasis treatment is similar to the process described in FIG. 11.

After the mucosal incision treatment, the operator switches the observation mode to the second narrow band light observation mode as in the case of the first embodiment, radiates narrow band light in the second narrow band light observation mode and then performs submucosal dissection (S4) and post-operation hemostasis treatment (S5).

As described above, according to the aforementioned two embodiments, the operator can perform appropriate mucosal incision in ESD of the large intestine and when bleeding occurs in the mucosal incision and treatment other than the mucosal incision, the operator can speedily perform hemostasis treatment.

The procedures described in the aforementioned two embodiments are applicable in ESD, and the aforementioned procedures are also applicable in EMR (endoscopic mucosal resection) or polypectomy that performs high-frequency snare treatment. When bleeding occurs in EMR or polypectomy, the operator can likewise switch the observation mode to the second narrow band light observation mode, check the bleeding point and perform hemostasis treatment.

Note that although the methods for endoscopic treatments according to the aforementioned two embodiments use a so-called frame-sequential endoscope apparatus using a monochrome image pickup device, a so-called simultaneous endoscope apparatus using a three primary color image pickup device or a complementary color image pickup device may also be used. In the case of a simultaneous endoscope apparatus, a plurality of light-emitting devices that emit their respective narrow band light beams may be used for an illumination apparatus and image acquiring timings may be controlled to prevent color mixing or whole wavelength information (reflected light) obtained from an object may be simultaneously detected.

Furthermore, according to the methods for endoscopic treatment according to the aforementioned two embodiments, the light source apparatus 4 uses a xenon lamp, but a light-emitting diode (LED) or laser diode (LD) may also be used to emit white light or band-limited light.

Furthermore, according to the methods for endoscopic treatments according to the aforementioned two embodiments, in the narrow band light observation mode, narrow band light having a predetermined peak wavelength is radiated onto a subject as band-limited light, but light including narrow band light having a predetermined peak wavelength and having a broad range or light including not only narrow band light having a predetermined peak wavelength but also wideband light in other wavelength bands may also be radiated onto the subject as band-limited light.

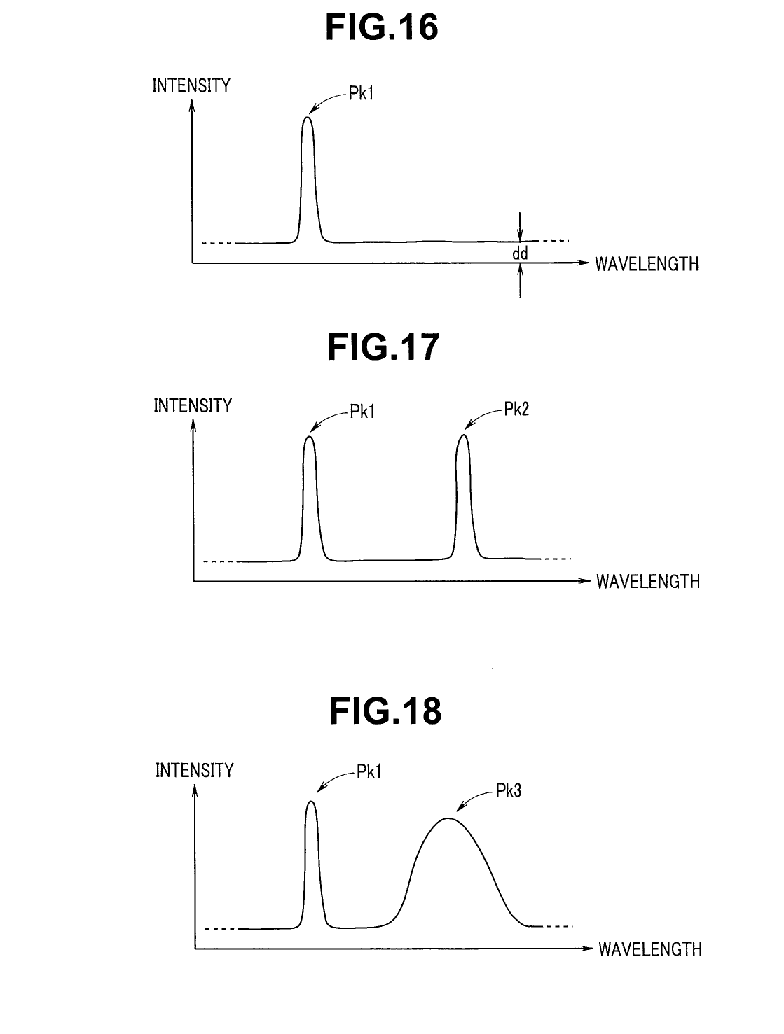

FIG. 16 to FIG. 18 are diagrams illustrating band-limited light. FIG. 16 is a diagram illustrating a relationship between wavelength and intensity of band-limited light including narrow band light having one predetermined peak wavelength and having a broad range. The band-limited light in FIG. 16 has a wavelength band including a peak Pk1, and has non-zero intensity dd in other wavelength bands.

FIG. 17 is a diagram illustrating a relationship between wavelength and intensity of band-limited light including narrow band light having two predetermined peak wavelengths and having a broad range. The band-limited light in FIG. 17 has a wavelength band including a peak Pk1 and a wavelength band including a peak Pk2 generated by a filter, and has non-zero intensity in other wavelength bands.

FIG. 18 is a diagram illustrating a relationship between wavelength and intensity of band-limited light including narrow band light having one predetermined peak wavelength and one wide band light. The band-limited light in FIG. 18 has a wavelength band including a peak Pk1 and wide band light including a peak Pk3, and has non-zero intensity in other wavelength bands. The light shown in FIG. 18 can be obtained by combining wide band light generated by fluorescent excitation light and narrow band light generated by a light-emitting diode (LED) or a laser diode (LD).

That is, not only narrow band light having a simple peak wavelength as described in the first and second embodiments but also the light described in FIG. 16 to FIG. 18 may be used as band-limited light for the methods for endoscopic treatment according to the aforementioned first and second embodiments.

The present invention is not limited to the aforementioned embodiments, but various modifications or changes or the like can be made without departing from the spirit and scope of the present invention.

What is claimed is:

1. A method for endoscopic treatment that performs treatment on a subject under an endoscope, the method comprising:
irradiating the subject with first narrow band light having a predetermined peak wavelength;
performing mucosal incision on a living tissue of the subject after irradiation with the first band-limited light;
radiating second band-limited light having a peak wavelength in spectral characteristics in a wavelength band closer to a long wavelength side than the first band-limited light after the mucosal incision; and
performing treatment other than the mucosal incision on the living tissue after radiation of the second band-limited light.

2. The method for endoscopic treatment according to claim 1, wherein in radiation of the first band-limited light, the first band-limited light is radiated to highlight a lesion range, and
in the mucosal incision, incision is performed on a periphery of the lesion range.

3. The method for endoscopic treatment according to claim 2, wherein in the treatment other than the mucosal incision, submucosal dissection or hemostasis treatment is performed on a peripheral region of the lesion range.

4. The method for endoscopic treatment according to claim 1, further comprising performing hemostasis treatment on a bleeding blood vessel of the living tissue under radiation of the band-limited light using an electric knife or hemostasis forceps.

5. The method for endoscopic treatment according to claim 4, further comprising switching from radiation of the band-limited light to radiation of the white light after the hemostasis treatment.

6. The method for endoscopic treatment according to claim 1, wherein the second band-limited light has a peak wavelength in spectral characteristics in a red band of a visible range between a wavelength band including a maximum value and a wavelength band including a minimum value in hemoglobin light absorption characteristics of the living tissue of the subject.

7. The method for endoscopic treatment according to claim 6, wherein the second band-limited light is light including narrow band light ranging from 585 nm to 630 nm.

8. The method for endoscopic treatment according to claim 1, wherein the living tissue is a large intestine mucous membrane.

9. A method for endoscopic treatment that performs treatment on a subject under an endoscope, the method comprising:
spraying a pigment over the subject;
radiating band-limited light having a predetermined peak wavelength after spraying of the pigment; and
a treatment step of performing submucosal dissection or hemostasis treatment on a living tissue of the subject after radiation of the band-limited light.

10. The method for endoscopic treatment according to claim 9, further comprising performing hemostasis treatment on a bleeding blood vessel of the living tissue under radiation of the band-limited light using an electric knife or hemostasis forceps.

11. The method for endoscopic treatment according to claim 10, further comprising switching from radiation of the band-limited light to radiation of the white light after the hemostasis treatment.

12. The method for endoscopic treatment according to claim 9, wherein the band-limited light has a peak wavelength in spectral characteristics in a red band of a visible range between a wavelength band including a maximum value and a wavelength band including a minimum value in hemoglobin light absorption characteristics of the living tissue of the subject.

13. The method for endoscopic treatment according to claim 12, wherein the band-limited light is light including narrow band light ranging from 585 nm to 630 nm.

14. The method for endoscopic treatment according to claim 9, wherein the living tissue is a large intestine mucous membrane.

15. A method for endoscopic treatment that performs treatment on a subject under an endoscope, the method comprising:
irradiating the subject with first band-limited light having a predetermined peak wavelength;
performing high-frequency snare treatment on a living tissue of the subject after irradiation with the first band-limited light; and
radiating second band-limited light having a peak wavelength in spectral characteristics in a wavelength band closer to a long wavelength side than the first band-limited light after the high-frequency snare treatment.

16. The method for endoscopic treatment according to claim 15, wherein the second band-limited light is light including narrow band light ranging from 585 nm to 630 nm.
